(19) United States
(12) Patent Application Publication
(10) Pub. No.: US 2010/0210878 A1
(43) Pub. Date: Aug. 19, 2010

(54) Release and Recovery from Aliphatic Primary Amines or Di-Amines

(75) Inventors: Esmaeel Naeemi, Lynnwood, WA (US); David G. O'Connor, North Bend, WA (US)

Correspondence Address: ASEMBLON, INC;JEFFREY B. OSTER -- LEGAL DEPARTMENT, 15340 NE 92ND ST, SUITE B, REDMOND, WA, 98052, US

(73) Assignee: Asemblon, Inc., Redmond, WA (US)

(21) Appl. No.: 12/707,658

(22) Filed: Feb. 17, 2010

Related U.S. Application Data
(60) Provisional application No. 61/153,147, filed on Feb. 17, 2009.

(51) Int. Cl. C07C 209/42; B01J 8/18

(52) U.S. Cl. 564/491; 422/141; 564/493

(57) ABSTRACT

There is disclosed a process for hydrogen release and chemical storage by dehydrogenating low molecular weight aliphatic amines and di-amines to produce their corresponding nitriles in a reactor system containing a hydrogen fractionation membrane (or sweep gas) to quickly remove any and all hydrogen generated during the dehydrogenation reaction. This disclosure further provides a process for hydrogen recovery using bi- and tri-functional amines that produce corresponding nitriles and high density hydrogen release.

### CROSS REFERENCE TO RELATED APPLICATION

This patent application claims priority from U.S. Patent Application 61/153,147 filed 17 Feb. 2009.

### TECHNICAL FIELD

This disclosure provides a process for hydrogen release and chemical storage by dehydrogenating low molecular weight aliphatic amines and di-amines to produce their corresponding nitriles in a reactor system containing a hydrogen fractionation membrane (or sweep gas) to remove any and all hydrogen generated during the dehydrogenation reaction. This disclosure further provides a process for hydrogen recovery using primary or di-amines that produce corresponding nitriles or di-nitriles and high density hydrogen release under specified reaction conditions.

### BACKGROUND

One major factor preventing widespread use of automotive fuel cells is the lack of a viable, on-board method for hydrogen storage. While many methods have been proposed, such as compressed hydrogen, metal hydrides, cryogenic hydrogen, reversibly hydrogenated liquids, and reactive chemical hydrides, each method has its own critical drawbacks (Ni, Energ. Explor. Exploit. 24:197-209, 2006; Ross, Vacuum 80:1084-1089, 2006; and Gray, Adv. Appl. Ceram. 106:25-28, 2007). In the area of hydrogen-storage liquids, optimal characteristics have included: (1) being capable of facile, clean and reversible degydrogenation; (2) having an enthalpy of dehydrogenation low enough that the dehydrogenation is thermodynamically favored at as low a temperature as possible (at least below 180° C.); (3) being in a liquid state and nonvolatile from −40° C. to the dehydrogenation temperature; (4) having a hydrogen storage capacity of at least greater than 6% by weight and 45 g H₂ per liter of liquid (Satyapal et al., Catal. Today 120:246-256, 2007); and (5) being stable against thermal or catalytic decomposition at operating temperatures.

Dehydrogenation enthalpy has been a problem preventing adoption of some early organic hysrogen storage liquids, such as benzene/cyclohexane (Cacciola et al., Int. J. Hydrogen Energy 9:411-419, 1984; Touzani et al., Int. J. Hydrogen Energy 9:929-936, 1984; and Klvana et al., Int. J. Hydrogen Energy 16:55-60, 1991). But the enthalpy of dehydrogenation for cyclohexane is so high that excessively high temperatures would be needed.

Another solution has been proposed in a study funded by DOE (U.S. Dept. of Energy) to Air Products to look at reversible dehydrogenation of nitrogen heterocycles (U.S. Pat. No. 7,101,530). Gaussian calculations showed that incorporation of at least one nitrogen atom into a ring can lower dehydrogenation enthalpy. Theoretically, enthalpy, entropy, Gibbs energy and optimal dehydrogenation temperature predicted that incorporation of nitrogen atoms into a single or multiple ring structure and the addition of electron donating groups would lower the temperature at which hydrogen can be released (Clot et al., Chem Commun. 2231-2233, 2007).

In this regard indoline over a Pd on carbon or Rh on carbon catalyst found dehydrogenation conversion after only 30 min of refluxing, a laboratory situation that does not lend itself to commercial applicability (Moores et al. New J. Chem. 30:1675-1678, 2006). However, indoline has too low a hydrogen content (1.7% by weight) to have commercial applicability.

### Dehydrogenation of Amines

Amines can be converted to nitriles over various catalysts, but the yield is affected by various side reactions that result in deamination and formation of hydrogen cyanide. As a result, continuous flow synthesis of nitriles by the selective oxidation of amines is a relatively inefficient process. Since the rate of these side reactions, however, is directly correlated with the concentration of hydrogen and nitriles at the catalytic sites, different methods have been investigated to quickly remove the hydrogen formed while oxidizing the amine, thereby reducing the chance of it reacting with the amine or nitrile. Since the product of interest for these reactions had been the formation of the nitrile and not hydrogen gas, it was hydrogen gas that had to be eliminated, often by using some other unsaturated compound to react with it. This was found to increase the nitrile yield.

The general reaction is represented by the following equation:

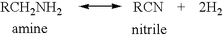

wherein R is any aliphatic or cyclo aliphatic moiety. Preferably, R is a methyl group so that the “product” of the reaction is acetonitrile and hydrogen. The problem initially encountered is that the nitrile was formed together with hydrogen, but only the nitrile was the desired product. More specifically, the nitrile formed was often immediately decomposed and re-hydrogenated back to the aliphatic amine. Initial solutions (in the 1920's and 1930's) was to vary the temperature of the reaction depending on the specific aliphatic amine or nitrile formed. But if the temperature was too low, an equilibrium was established that had inadequate conversion of the amine to the nitrile due to too much rehydrogenation of the nitrile back to the amine. Conversely, if the temperature was too high, the amine and the nitrile both decomposed. The breakthrough came in U.S. Pat. No. 2,388,218 (the disclosure of which is incorporated by reference herein), filed in 1943, where a hydrogen acceptor was added to be hydrogenated, particularly, hydrogenating across olephinic double bonds. In that way, the hydrogen formed is used to hydrogenate other molecules and not the newly formed nitrile moiety. This improved yields of nitriles.

Subsequent developments improved the basic dehydrogenation reaction to now generate water as a by-product to soak up the hydrogen generated with an oxygenator added to the reaction and this process has become the standard for industrial production of nitriles, particularly acetonitrile. Amines have been found to oxidize to nitriles when reacted with K₂S₂O₈ over NiSO₄ (Yamazaki and Yamazaki, Bull. Chem. Soc. Jpn. 63:301-303, 1990), or in gas flow reactions with halide cluster catalysts such as [(Mo₆Br₈)(OH)₄(H₂O)₂] and [(Ta₆Cl₁₂)Cl₂(H₂O)₄]₄H₂O (Yamazaki and Yamazaki, Bull. Chem. Soc. Jpn. 63:301-303, 1990). Although such reactions have been found and used to form nitriles, such reactions have been conducted as oxidation reactions that form water as the by-product. As a result, such methods of oxidizing amines to form nitriles cannot be used to release and collect hydrogen gas.

### Bi-Functional Amines

Dehydrogenation of single-functional aliphatic amines is a preferred reaction to form nitriles, although the reverse reaction (hydrogenation of aliphatic nitriles to form an aliphatic amine is preferred. While non-catalytic thermal dehydrogenation of organic compounds is known, the use of such methods is limited due to extensive undesirable side effects which take place. Thus, catalytic processes have been developed in order to minimize side reaction activity and improve conversion and selectivity to desired products. But such reactions have focused on the final product and minimizing undesired product generation. The hydrogen generated during any dehydrogenation reaction was considered waste.

Common catalysts for dehydrogenation reactions include Group VIII metals (and alloys and combinations). More particularly, various noble metals are preferred. But some catalysts do not have a long life-span as effective catalysts. For example, platinum/tin/zinc aluminate catalysts are highly active and selective for dehydrogenation reactions (particularly paraffins) but such catalysts quickly lose their activity and need to be regenerated at periodic intervals.

Primary aliphatic amines (such as ethyl-amine) and di-amines (such as 1,3-diamino propane) are commonly used as intermediates during synthesis of larger molecules. For example, hexamethylene diamine is useful as an intermediate in the production of Nylon (U.S. Pat. No. 3,414,622) with the initial reaction being a dehydrogenation reaction. 1,3-Diamino propane is a compound that imparts desirable qualities to textile resins when used in the form of propylene urea and is useful as an intermediate in the preparation of sequestering agents, herbicides and polyamides for use in textile fibers. Often, 1,3-diamino propane is made by converting alkylene bisoxdipropinitrile to 1,3-diamino propane by heating an alkylene bis-oxydiproppionitrile with a hydrogenation catalyst under hydrogen and ammonia. But a byproduct of secondary amines often results.

### Hydrogen-Selective Membranes

Membranes are thin, perm-selective materials that separate desired chemical species from a mixture of chemical species. Hydrogen-selective membranes in particular are used in reactors or devices to separate hydrogen generated from other gaseous mixtures that have potential to poison catalysts in fuel cells (such as Pt catalysts commonly used in PEM type hydrogen fuel cells).

Specifically, gas molecules passing through porous alumina membranes with pore sizes of from about 5 nm to hundreds of nanometers follow the Knudsen diffusion mechanism with a binary selectivity between molecules A and B (S_{AB}), which is proportional to the square root of the inverse ratio of the molecular weights

$$( {S}_{AB} = \sqrt{\frac{{M}_{B}}{{M}_{A}}} ) ,$$

where M_{A} and M_{B} are the molecular weights of gas molecules A and B, respectively (Burggraaf and Cot, “Fundamentals of inorganic membrane science and technology,” Elsevier, 1996; p. 331). This provides selectivities of H₂/CH₄=2.8, H₂/N₂=3.7, and H₂/CO₂=4.7. Since the Knudsen diffusion selectivities are too low to produce pure hydrogen, various surface modification techniques like sol-gel, chemical vapor deposition, sputtering, and electroless plating have been applied to improve the properties of mesoporous and macroporous supports to overcome these low selectivities.

Hydrogen-selective membranes have been used in industrial processing, petroleum refining and more recently for hydrogen fuel cells (to provide efficient DC power) for purified hydrogen. Palladium membranes have been used because palladium (Pd) has high selectivity for hydrogen over other gas molecules. Various methods have been used to prepare palladium membranes, such as, metal-organic chemical vapor deposition (MOCVD) (Yan et al., Ind. Eng. Chem. Res. 33:616, 1994), sputter deposition (Jayaraman et al., J. Membr. Sci. 99:89, 1995), electroless plating (Roa et al., Chem. Eng. J. 93:11, 2003), and a combination of electroless plating and electroplating (Tong et al., Ind. Eng. Chem. Res. 45:648, 2006).

Electroless plating of palladium is presented in reaction 1. This process is an autocatalytic reaction with a reducing agent, hydrazine (N₂H₄).

2Pd(NH₃)₄²⁺+N₂H₄+4OH⁻→2Pd+8NH₃+N₂+4H₂O (1)

It is desirable to have palladium membranes with both high hydrogen permeance and selectivity. The mechanism of hydrogen transport through a palladium membrane is based on the dependence of hydrogen flux on pressure difference as given in equation (2)

$$J = \frac{D ( {P}_{h}^{n} - {P}_{l}^{n} )}{l} \qquad ( 2 )$$

In this equation J is the hydrogen flux, D is the hydrogen diffusion coefficient, l is the film thickness, Pₕ is the partial pressure of hydrogen in the feed, and Pₗ is the partial pressure of hydrogen in the permeate. Hydrogen transport through a palladium membrane can be categorized into three regions depending on the value of n. When n=0.5 the expression is known as Sievert's law and the transport is limited by bulk diffusion through the palladium layer, when n=1, the transport is limited by mass transport to the surface or by a process at the surface itself, when 0.5<n<1, the transport is limited by a combination of bulk diffusion and the surface process (Wu et al., Ind. Eng. Chem. Res. 39:342, 2000).

Therefore, there is a need in the art to develop improved hydrogen storage molecules and formulations and reactor devices to optimally utilize chemical reactions to quickly remove

### SUMMARY

This present disclosure provides that several catalysts, including cobalt and its various oxides, iron oxide, nickel oxide, and chromium oxide can dehydrogenate several types of alkylamines to their respective nitriles. In addition, this disclosure provides that and under the specified conditions, the disclosed process removes hydrogen gas. The hydrogen gas is removed in a separator, filtered, and fed directly to either fuel cell or engine. The amine is recycled, as single pass conversion through the reaction is relatively low, and the nitrile is collected and stored for later rehydrogenation.

The present disclosure provides a reactor system for capturing hydrogen as a pure gas from the dehydrogenation of primary aliphatic amines or di-amines before the dehydrogenation reaction product of the dehydrogenation of can be re-hydrogenated. More specifically, the present disclosure provides a reactor system for dehydrogenating primary aliphatic or di-amines to their corresponding nitriles, comprising:

(a) a flow-through reactor having an inner reactor located within and extending outside of an outer chamber;

(b) an inner reactor comprising a catalyst bed, an inlet and an outlet, and having a first wall composed of a hydrogen membrane in that portion of the inner reactor located within the outer chamber, and a second wall of an impermeable material in that portion of the inner reactor located outside of the outer chamber, wherein the inlet further comprises a means for vaporizing a liquid to form a gaseous state prior to entering the catalyst bed;

(c) an outer chamber having an outlet, inner walls and outer walls and surrounding the catalyst bed portion of the inner reactor, wherein the inner walls are the first wall of the inner reactor, and wherein the outlet further comprises a vacuum to pull through purified hydrogen formed in the inner reactor.

Preferably, the dehydrogenation catalyst in the inner reactor is selected from the group consisting of heterogeneous or homogeneous Group VIII metals, Rh, Pt, Ru, Au, Pd, cobalt, cobalt oxide, iron oxide, nickel oxide, chromium oxide, and alloys and combinations thereof. Preferably, the dehydrogenation catalyst is Co or Co oxide when the amine is a primary amine or Rh or Pt when the amine is an alkyl di-amine. Preferably, the outer chamber or the inner reactor is surrounded by a resistive heating element to provide sufficient temperature to catalyze the dehydrogenation reaction. Preferably, the alkyl di-amine is selected from the group consisting of 2-(aminomethyl)propane-1,3-diamine, propane-1,3-diamine, propylamine, ethylamine, butyl amine, propane-1,3-diamine, ethane-1,3-diamine, butane-1,3-diamine, pentane-1,3-diamine, isopropyl-1,3-diamine, and combinations thereof.

The present disclosure further provides a reactor system for dehydrogenating primary aliphatic mono- or di-amines to their corresponding nitriles, comprising:

(a) a flow-through reactor having a circumferential outer reactor located surrounding an inner chamber;

(b) an outer circumferential reactor comprising a catalyst bed, an inlet and an outlet, and having an inner wall composed of a hydrogen membrane in that portion of the outer circumferential reactor located surrounding the inner chamber, and an outer wall of an impermeable material in that portion of the inner reactor located outside of the inner chamber, wherein the inlet further comprises a means for vaporizing a liquid to form a gaseous state prior to entering the catalyst bed;

(c) an inner chamber having an outlet and outer walls, wherein the outer walls are the inner walls of the circumferential reactor, and wherein the outlet further comprises a vacuum to pull through purified hydrogen formed in the outer circumferential reactor.

Preferably, the dehydrogenation catalyst in the outer circumferential reactor is selected from the group consisting of heterogeneous or homogeneous Group VIII metals, Rh, Pt, Ru, Au, Pd, cobalt, cobalt oxide, iron oxide, nickel oxide, chromium oxide, alloys of the foregoing and combinations thereof. Preferably, the outer circumferential reactor is surrounded by a resistive heating element to provide sufficient temperature to catalyze the dehydrogenation reaction,

The present disclosure further provides a process for dehydrogenating an aliphatic mono- or di-amine to its corresponding mono mono- and di-nitriles, comprising:

(a) providing a mono- and di-amine or a mixture thereof in a vapor form to a reactor having a dehydrogenation catalyst, an inlet and an outlet, wherein the mono- or di-amine or the mixture thereof is provided through the inlet and wherein the dehydrogenation catalyst is selected from the group consisting of Rh, Pt, Ru, Au, Pd, cobalt, cobalt oxide, iron oxide, nickel oxide, chromium oxide, alloys of the foregoing and combinations thereof;

(b) providing sufficient heat to dehydrogenate the mono- or di-amine into its corresponding mono- or di-nitriles and hydrogen gas;

(c) physically removing the hydrogen gas through a fractionating hydrogen membrane or by utilizing an inert sweep gas; and

(d) recovering the mono- or di-nitriles and the mixtures thereof formed by condensing the vapor in the outlet and recovering condensed liquid.

Preferably, the aliphatic di-amine or mono-amine is selected from the group consisting of 2-(aminomethyl)propane-1,3-diamine, propane-1,3-diamine, propylamine, ethylamine, butyl amine, propane-1,3-diamine, ethane-1,3-diamine, butane-1,3-diamine, pentane-1,3-diamine, isopropyl-1,3-diamine, and combinations thereof. Preferably, the sweep gas is selected from the group consisting of He, Ar, and combinations thereof.

### BRIEF DESCRIPTION OF THE FIGURES

FIG. 1 shows an outside and side view of the prototype reactor.

FIG. 2 shows a prototype reactor in a cut-out view showing how the reactor is assembled.

FIG. 3 shows a liquid chromatography spectrum of the output of a reaction of 1,3-diaminopropane in the top panel and a mass spectrum of pure 1,3-diaminopropane in the bottom panel.

FIG. 4 shows conversion (yield of propionitrile) versus temperature curve. Yield of propionitrile was calculated as (propionitrile in product stream)/(propylamine in inlet stream). Apparent activation energy calculated from these results, were 129 kJ/mol and 215 kJ/mol for commercial cobalt and nickel particles, respectively.

### DETAILED DESCRIPTION

The present disclosure provides that various alkylamines are converted to their respective nitriles while recovering hydrogen. The process has been tested for aminoalkanes, over cobalt, cobalt(II)oxide, cobalt(III)oxide, iron(II)oxide, iron(III)oxide, chromium oxide, and nickel oxide as the catalyst candidates. Commercial cobalt metal catalysts were purchased from Fluka, other catalysts were purchased from Aldrich. The gases employed were H₂ (Airgas, Grade 5, 99.99%), He (Airgas, Grade 5, 99.99%), O₂ (Airgas, UHP Grade, 99.99%) and N₂ (Airgas Grade 5, 99.99%).

Microwave cobalt oxide was made according to U.S. Pat. No. 7,309,479, the disclosure of which is incorporated by reference herein. Briefly, cobalt metal powder (5 grams) was placed in a ceramic crucible and placed in a microwave oven for 3 minutes. The power of the microwave was set to 950 W. Upon generating the microwave, the cobalt metal powder started glowing red-hot within a minute. The microwave heating of the sample continued for 3 minutes. After completion the sample was crushed and used without further treatment. Samples were analyzed by GC/MS (Hewlett-Packard, 5890-5972A equipped with a 0.25 mm i.d.×30 m fused silica capillary column).

Propylamine was the alkylamine used to test catalysts. Propylamine is a liquid at room temperature. The dehydrogenation reaction produces two moles of hydrogen gas by oxidizing propylamine into propionitrile. Since the desired product, the two moles of hydrogen gas, are formed when propionitrile is formed, propionitrile was used as an indication of the dehydrogenation reaction. This was because propionitrile could easily be quantified with respect to the propylamine in a GC/MS. The catalysts that showed some promise were tested in a larger packed bed, which incorporated a qualitative hydrogen gas detector in the exit stream, allowing for both a direct observation of hydrogen gas formation, and an indirect one by the production of propionitrile. In a final reactor setup, a mass flow meter was used in series with the qualitative hydrogen gas detector to quantitatively measure the hydrogen gas produced.

Catalysts first underwent a qualitative screening in which ˜0.5 g of each catalyst was packed into a glass tube with an inner diameter of 0.93 mm, making a small, packed bed reactor, and placed in a controllable heated housing under a constant flow of helium (sweep gas) at 10 psig. The heated housing was adjusted to various temperatures, ranging from 200° C. to 300° C., and at each temperature, 0.1 ul of propylamine was injected into the inlet of the glass tube, where it was carried by the helium sweep gas, flowing at a range of 1-2 ml/min, depending on the packing of each catalyst. The exit stream of the reactor was connected to the GC/MS, where it was analyzed for the starting material, propionitirle (desired byproduct), and any other byproducts.

The results were analyzed by, for example, the area of the propionitrile peak versus the total area of other compounds. This provided an approximate percent conversion and the number of peaks other than propylamine and propionitrile were used to give a high, medium, or low selectivity label to each run. A high selectivity label indicated only propionitrile and propylamine were observed. A medium selectivity indicated up to two additional byproducts. A low selectivity indicated more than two additional byproducts. Of all the catalysts tested, only a few were selected for the larger scale testing, and they are listed in the following table.

| | 200° C. | 250° C. | 280° C. | 300° C. |
|---|---|---|---|---|
| Cobalt | | | | |
| Conversion | No Conversion | No Conversion | 63% | 78% |
| Selectivity | No Conversion | No Conversion | Medium | High |
| Cobalt Oxide | | | | |
| Conversion | No Conversion | No Conversion | No Conversion | 47% |
| Selectivity | No Conversion | No Conversion | No Conversion | Medium |
| Cobalt Micro | | | | |
| Conversion | 7% | 72% | 72% | 67% |
| Selectivity | High | Medium | High | Medium |
| Nickel | | | | |
| Conversion | 40% | 60% | 65% | 60% |
| Selectivity | High | High | Medium | Low |

After the screening process, the catalysts listed in Table 1 were tested in a larger packed bed reactor setup. 0.5 Gm of catalyst was packed in ¼ inch ID tube with glass wool at both ends of the catalyst. Each catalyst was heated to a specified reaction temperature. The temperature at the catalyst surface was measured by a thermocouple and controlled by a temperature controller. Propylamine was delivered to the reactor by a dual piston pump and the pump controlled inlet volumetric flow rate. Propylamine was vaporized prior to entering the reactor and vapor temperature was controlled and set at catalyst temperature. The product stream exiting the reactor was condensed to room temperature using ice cold water. Inlet stream and product stream were analyzed for propionitrile by GC/MS to calculate conversion. A qualitative hydrogen gas detector was used to identify the production of hydrogen gas in the reaction. The reactivity of each catalyst was measured at various temperatures. The temperature of reaction was varied from 250° C. to 350° C. The inlet flow rate was 0.125 ml/min that corresponds to a space velocity of 4181 ml/hr/g of catalyst. Space velocity is defined as inlet gas volumetric flow rate divided by weight of catalyst.

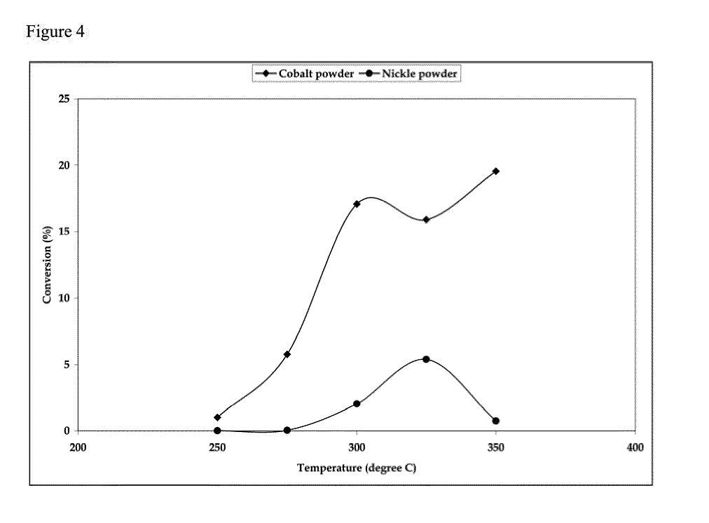

The results of the studies are presented in FIG. 4. Yield of propionitrile was calculated as (propionitrile in product stream)/(propylamine in inlet stream). FIG. 4 shows conversion (yield of propionitrile) versus temperature curve. Apparent activation energy calculated from these results, were 129 kJ/mol and 215 kJ/mol for commercial cobalt and nickel particles, respectively.

Since cobalt oxide performed well enough in the packed bed reactor, it was used as a catalyst in the large, monolith reactor. This reactor consisted of an 18 inch long stainless steel, tubular housing with a 4.5 inch inner diameter, containing two 900 cpsi (cells per square inch) and two 1000 cpsi monoliths. The reactor was heated by a series of band heaters wrapped around the outside of the reactor housing, while the temperature was controlled based on a series of thermocouples set inside the reactor. A pump was used to push the liquid fuel (an alkylamine, specifically propylamine) from a reservoir, through a series of vaporizers, and into the reactor. The first vaporizer was used to vaporize the liquid fuel, while the second was used to heat the fuel vapor to the same temperature as that of the catalyst. The exiting vapor from the reactor was passed through a heat exchanger, lowering the overall temperature of the exiting stream to approximately 30° C. At this point, the mixture of vapor and liquids was passed through a separator (i.e., a 2 liter closed container that has a ¼ inch liquid outlet at the lower part (returns the liquid back to the reservoir) and a ¼ inch gas outlet on the top for the gases), where the liquids reentered the reservoir and the vapors passed into a series of scrubbers, removing all traces of organic compounds from the produced hydrogen gas. The final, purified stream of hydrogen gas was then passed through a qualitative hydrogen detector, indicating ˜100% purity, and a mass flow meter, indicating ˜4.3 L/min of gas flow. This hydrogen was then directly fed into a 4-stroke Honda engine with a 25 cc displacement modified to run on hydrogen gas.

Cobalt and cobalt oxide both showed promising results as catalysts for the dehydrogenation reaction in the various reactors disclosed herein. Cobalt oxide was tested in two forms, one being the commercially available cobalt oxide (Fluka) and the other, the microwave cobalt oxide described herein. Both cobalt oxides were effective in the preliminary screening, as well as in the larger packed bed reactor. A larger monolith reactor (Hypercat) tested one form of cobalt oxide, deposited on the monolith (deposited by wash coating with 3-5% by weight loading).

Nickel showed high conversion and selectivity at relatively mild temperatures, such as approximately 250° C., during the screening process, but performed poorly in the larger scale, where the selectivity dropped significantly. While this could be due to a number of factors, and without being bound by theory, nickel has a high affinity for hydrogen and was adsorbing the hydrogen gas produced and thereby catalyzing many other side reactions. A small amount of the nickel catalyst was placed in the glass tube setup used for screening the catalysts. This setup initially required a helium sweep gas to continuously flow through the catalyst, carrying the injected propylamine and all reaction products through the reactor. However, the helium gas was now replaced with a hydrogen sweep gas, so that hydrogen was now constantly available at the catalysts to interfere with the reaction. Under helium flow, nickel had performed highly selectively in producing propionitrile. Under hydrogen flow, however, no propylamine or propionitrile was detected, while many other side products were. Therefore, and without being bound by theory, since nickel is often used as a hydrogenation catalyst, the presence of hydrogen changed nickel's catalytic activity. Therefore, the present disclosure requires a functioning means for removing hydrogen as soon as it is formed. Accordingly, if the hydrogen is not removed form the reactor it causes parallel and side reactions.

As for the catalysts that did not pass the initial screening phase, including copper, chromium, iron, and their oxides, the results either indicated low overall activity or, as in the case of copper, high activity but extremely low selectivity.

The fact that propylamine, a mono amine, was tested confirmed the potential for alkylamines in general to undergo this oxidation process and release hydrogen gas. However, the other proposed alkylamines may perform at different rates when tested with each catalyst, and so they will each be tested in a similar fashion. One important question, however, was whether alkylamines and alkylnitriles reacted with to form the observed side products by heat alone, and so several tests were performed where a known mixture of propylamine and propionitrile were both heated to reactor temperatures and their composition monitored by GC/MS. Also, they were passed through a packed bed containing powdered glass, to simulate the reactor without the catalyst. In both cases, the composition of the mixtures remained the same, indicating that no reaction was taking place without the presence of a catalyst. Therefore, it can be concluded that propylamine, by passing over the cobalt and cobalt oxide catalysts at temperatures ranging from 250° C. to 300° C., formed propionitrile as the major product by releasing hydrogen gas, which could then be used to power a motor by combustion. Also, the propylamine and propionitrile underwent the various reactions to form the minor, side products in the presence of those catalysts. Minor products, such as those mentioned in earlier publications regarding the preparation of alkylnitriles by alkylamine oxidation, were also observed, including cyanide and ammonia. However, since propionitrile was not the desired compound, the reaction yield was lowered to increase selectivity, reducing these byproducts, and any unreacted propylamine was recycled along with the other liquids reentering the reservoir. To improve the process further, a selective membrane can be used to help remove hydrogen gas from the reaction stream, eliminating its role in side reactions.

### Reactor

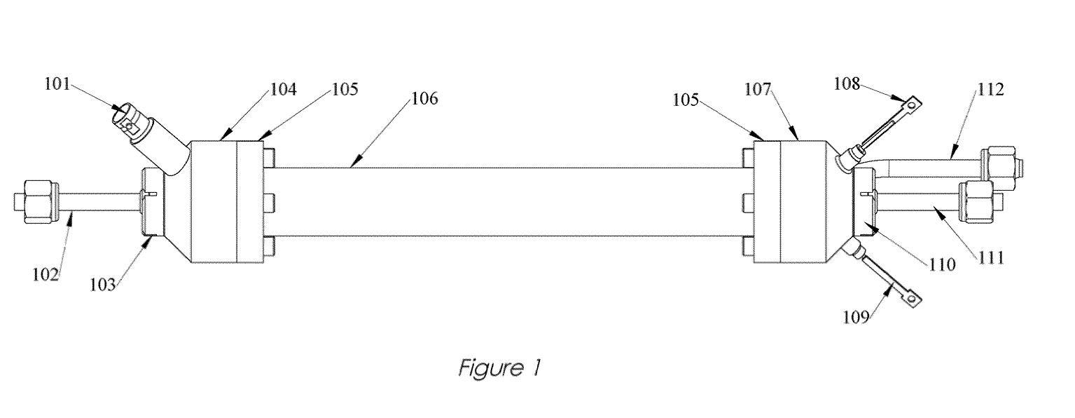

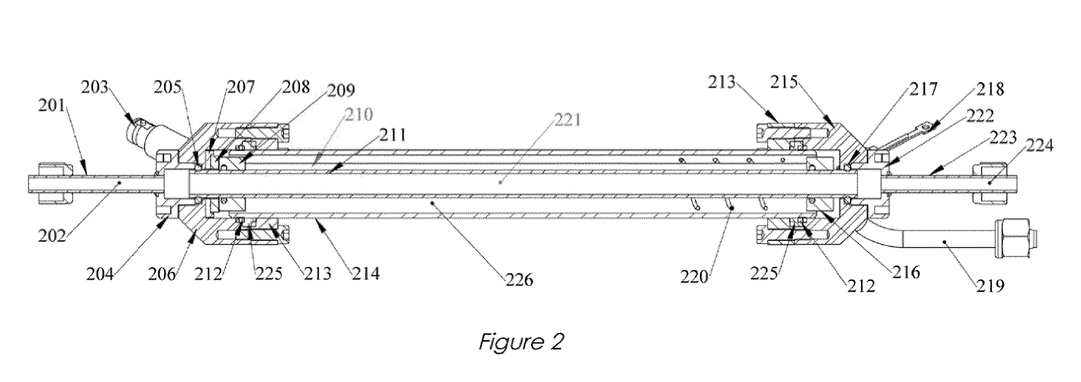

With regard to FIG. 1 (with one reference to FIG. 2), power comes into the reactor through a power input connector 101, so as to provide current to a plurality of heaters that heat the outer surface of the membrane separator tube 211. Vaporized fuel (primary amine) flows into the disclosed reactor through a vapor fuel inlet tube 102 to deliver fuel, in a vapor form, to an inside volume of the membrane separator tube 211. An inlet cap flange 103 holds the vapor fuel inlet tube 102 to an inlet tube cap 104 and the inlet cap flange seals the vapor fuel inlet tube 102 using, for example, and O-ring. The inlet tube cap 104 holds the membrane separator tube 211, outer reactor tube 106, power input connector 101, and tube heater 210 into place on the inlet side. The inlet tube cap 104 also provides sealing surfaces between the membrane separator tube 211 and an outer reactor tube 106. A cap flange clamp 105 holds the outer reactor tube 106 to the inlet cap flange 103. The outer reactor tube 106 defines the length of the reactor in addition to providing the chamber for capturing hydrogen. An outlet tube cap 107 holds the membrane separator tube 211, outer reactor tube 106, power input connector 101 and tube heater 210 into place on the outlet side of the reactor. The outlet tube cap 107 also provides sealing surfaces between the membrane separator tube 211 and the outer reactor tube 106.

A positive thermocouple feed-through 108 provides electrically connecting to the positive lead of a thermocouple 220, which is in contact with the membrane separator tube 211. A negative thermocouple feed-through 109 provides electrically connecting to the negative lead of a thermocouple 220, which is in contact with the membrane separator tube 211. A recirculation outlet flange 110 holds s recirculation outlet tube 111 to the outlet tube cap 107. The recirculation outlet flange 110 also provides for sealing using, for example, an O-ring. The recirculation outlet tube 111 provides for removing un-reacted fuel from the reactor. A hydrogen outlet tube 112 is where hydrogen is produced.

Further with regard to the embodiment in FIG. 1, power comes into the reactor through a power input connector 101 to provide current to the heaters that heat the outer surface of the membrane separator tube 211. A vapor fuel inlet tube 102 provides vaporized fuel flows into the reactor to deliver fuel to the inside volume of the membrane separator tube 211. An inlet cap flange 103 holds the vapor fuel inlet tube 102 to the inlet tube cap 104 and provides for sealing the vapor fuel inlet tube 102 to the inlet cap flange 103 such as with an O-ring. A cap flange cap 105 holds an outer reactor tube 106 to the inlet cap flange 103. The outer reactor tube 106 defines the length of the reactor and captures hydrogen generated in the dehydrogenation reaction. An outlet tube cap 107 holds the membrane separator tube 211, outer reactor tube 106, power input connector 101, and tube heater 210 into place on the outlet side. The outlet tube cap 107 also provides sealing surfaces between the membrane separator tube 211 and the outer reactor tube 106. A positive thermocouple feed through 108 and a negative thermocouple feed through 109 provide provides electrical connects to a positive or negative lead of the thermocouple which is in contact with the membrane separator tube. A recirculation outlet flange 110 holds a recirculation outlet tube 111 to the outlet tube cap 107. The recirculation outlet flange 110 also provides for sealing the recirculation outlet tube 111 to the outlet tube cap 107, for example, using an O ring. The recirculation outlet tube 111 also removes un-reacted fuel from the reactor. The hydrogen outlet tube 112 is where hydrogen is produced.

With regard to FIG. 2, inlet tube 201 is welded to inlet cap flange 204. Outlet tube 233 is welded to outlet cap flange 222. Reactor outer tube flanges 225 are welded to a reactor outer tube 214 to form a gas tight seal a few millimeters from the ends of a reactor outer tube 214. The weld bead is only on the outside surface of the flange closest to the ends of the tube. Clamps 213 are used to clamp against reactor outer tube flanges 225 and seal with O-rings 212 to an inlet flange 206 on one end and an outlet flange 215 on the other end. The membrane reactor tube 211 is positioned down the center of the reactor outer tube 214 so that the ends of the membrane reactor tube 211 are approximately equal distance from the ends of both the inlet flange 206 and the outlet flange 215.

The membrane reactor tube 211 is held in place and sealed to the reactor with O-rings 212. The O-ring 212 and membrane reactor tube 211 are clamped and sealed at the inlet flange 206 end of the reactor by inlet cap flange 204. The O-ring 212 and the membrane reactor tube 211 are clamped and sealed at the outlet flange 215 end of the reactor by an outlet cap flange 222. An outer diameter 203 of power connector 101 is welded to an inlet flange 104, 206 to form a gas tight seal. The center electrode of the power connector 101, 203 passes through an insulator 207 and couples to an input power electrode 208 where it is welded into place to form a solid electrical connection.

A positive thermocouple feed-through 108 is welded to an outlet tube cap 107. Negative thermocouple feed-through 109 is also welded to the outlet tube cap 107, 215. Both welds form a gastight seal. Hydrogen outlet tube 112, 220 is welded to the outlet tube cap 107, 215 to form a gastight seal. Heater electrodes 209 are positioned at equal distances from the ends of the membrane reactor tube 211. Four graphite carbon rods 210 are placed around the outside perimeter at about 90 degrees from each other so that the outside circumference of the membrane reactor tube 211 and the graphite carbon rods 210 are touching each other. The heater electrodes 209 secure the graphite carbon rods 210 in place and provide electrical contact to the rods. The rods are secured to the heater electrodes 209 are with set screws on either end of the graphite carbon rod 210. Electrical connection to the heater electrodes 209 is provided by connecting one end of the outlet flange 215 to an end to the heater electrode 209 by means of a screw. The other end of the inlet flange 206 end is connected to an input power electrode 208 by means of a screw.

Heater temperature is monitored through a thermocouple 220. A thermocouple 220 positive lead is connected to the positive thermocouple feed-through 108. A thermocouple 220 negative lead is connected to the negative thermocouple feed-through 109. The thermocouple 220 is secured to the outer circumference of the membrane reactor tube 211 in approximately the middle of its length between the graphite carbon rods 210. Vaporized fuel enters the reactor through tube 201 and passes thru the inlet cap flange 204 and then into the active reactor volume 221. There, the vapor comes in contact with the inner surface of the reactor membrane tube 211 where the fuel separates into hydrogen gas and spent fuel and passes through a hydrogen membrane within the tube wall and into a space 226 between the outer membrane tube 211 and the inner wall of the outer reactor tube 214. Hydrogen and reaction byproducts (such as spent fuel in the form of various nitriles) then exit the reactor through tube 219. Any unreacted fuel exits the inner volume 221 of the membrane reactor tube 211 through tube 223 where it is recirculated back to the feedstock after condensing.

### Dehydrogenation of Aliphatic Amines

The present disclosure provides a series of primary amines and di-amines, which catalytically dehydrogenate at elevated temperatures to form their corresponding nitriles or imines and produce hydrogen gas. Most preferred catalysts are Rh, Pt, Ru, Au or Pd mixed or pure anchored on high surface area substrate. The dehydrogenated products can be rehydrogenated to the original starting amine by hydrogenation over Pd/C catalyst at a range of concentrations.

### Example 1

This example illustrates the dehydrogenation reaction of 1,3-diaminopropane with Rh—Pt catalyst on gamma aluminum to form acetonitrile.

Pt—Rh bimetallic catalyst on alumina (“Catalyst A”) was synthesized according to Kariya et al. (Applied Catalysis A: General 247:247-259, 2003) by dissolving 724 mg chloroplatinic acid (H₂PtCl₆) in 900 ml water to form a catalyst solution. 371 mg of rhodium chloride (RhCl₃) was added to the catalyst solution and stirred for 5 minutes. Then, 6000 mg of gamma-alumina was added to the catalyst solution and stirred for 24 hours. The catalyst solution was filtered and the gamma-alumina powder was washed with DI water. The gamma-alumina powder was vacuum dried for 24 hours. The catalyst powder was then reduced by flowing hydrogen gas (50 ml/min) at a ramp temperature (0.73° C./min) of 25° C. to 200° C. in 2 hour. ICP/MS results show the loading of 0.49 wt/wt % of Rh and 0.50 wt/wt % of Pt.

Ethylamine and 1,3-diaminopropane were purchased commercially (Sigma-Aldrich).

The dehydrogenation reaction was performed and monitored by an HP GC 5890 series II equipped with HP5971 mass detector. The samples were run with the same procedure: initial temperature at 40° C., which was held for 3 minutes. The temperature was then increased at a rate of 10/min until reaching 120° C. The temperature was then ramped at a rate of 25° C./min until reaching 260° C. were it is held for 8 minutes. There was an injection of 1 microliter for every sample analyzed on the GC.

The inlet liner of gas chromatograph (78 mm×0.93 mm-id) was packed with catalyst A (8.2 mm³, 0.1-5 g). The liner was placed in the inlet port of the instrument and it was heated to 280° C. The desired starting molecule to be tested was placed in a vial equipped with septum. The headspace of the septum was vacuumed. A gas-tight syringe was used to extract 0.1-5 μl of the headspace gases and inject the extracted gases into the GC/MS. Helium gas (8 psi) pushed the sample through catalyst into the GC column. The reaction takes place in the liner and was directly monitored by the mass detector.

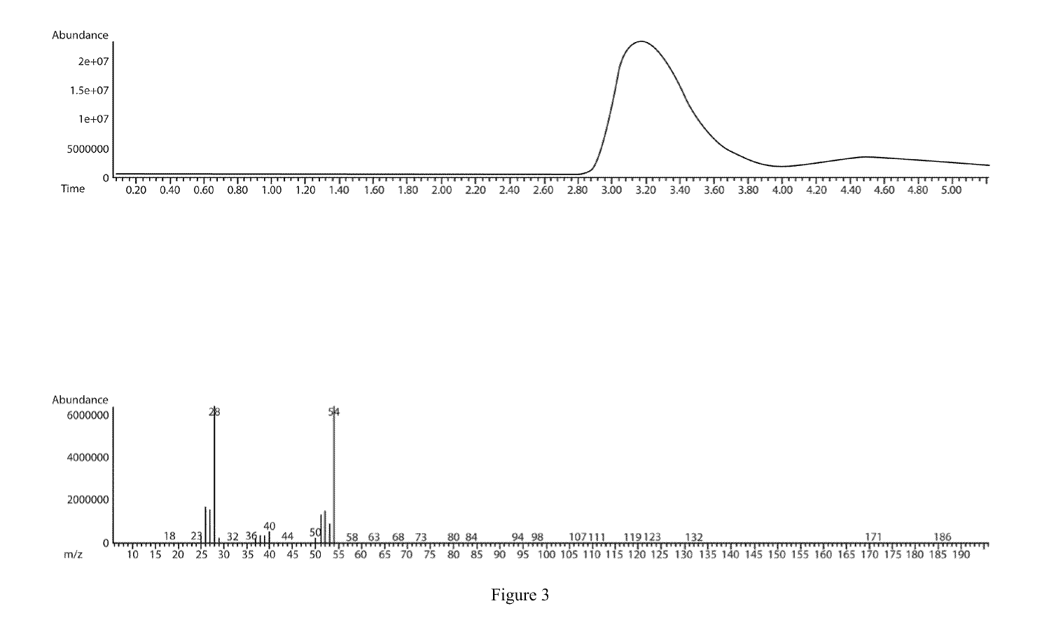

The mass spectrum of pure 1,3-diaminopropane (FW=74) did not show a parent peak at m/z=74. Instead, it showed a major fragment at m/z=57 and base peak at m/z=30. The m/z=57 peak was due to the loss of ammonia (NH₃=17) which took place in the ion source (FIG. 3, top panel). Mass spectrum of 1,3-diaminopropane over the Rh—Pt catalyst A showed was fully converted to several dehydrogenated compounds (FIG. 3, bottom panel. Specifically, an effluent at 2.6 minute had a m/z=54, which corresponds to propinonitrile, a mono dehydrogenation of one of the amine moieties. Close analysis of the other effluent and their corresponding spectrums showed either nitrile or alkyne. Seeing dehydrogenated products under our experimental conditions where there is no possibility of dehydration or dehydrohalogenation suggests production of hydrogen gas as one of the by-products.

We claim:

1. A reactor system for dehydrogenating primary aliphatic or di-amines to their corresponding nitriles, comprising:
(a) a flow-through reactor having an inner reactor located within and extending outside of an outer chamber;
(b) an inner reactor comprising a catalyst bed, an inlet and an outlet, and having a first wall composed of a hydrogen membrane in that portion of the inner reactor located within the outer chamber, and a second wall of an impermeable material in that portion of the inner reactor located outside of the outer chamber, wherein the inlet further comprises a means for vaporizing a liquid to form a gaseous state prior to entering the catalyst bed;
(c) an outer chamber having an outlet, inner walls and outer walls and surrounding the catalyst bed portion of the inner reactor, wherein the inner walls are the first wall of the inner reactor, and wherein the outlet further comprises a vacuum to pull through purified hydrogen formed in the inner reactor.

2. The reactor system for dehydrogenating primary aliphatic or di-amines to their corresponding nitriles of claim 1, wherein the dehydrogenation catalyst in the inner reactor is selected from the group consisting of heterogeneous or homogeneous Group VIII metals, Rh, Pt, Ru, Au, Pd, cobalt, cobalt oxide, iron oxide, nickel oxide, chromium oxide, and alloys and combinations thereof.

3. The reactor system for dehydrogenating primary aliphatic or di-amines to their corresponding nitriles of claim 1, wherein the dehydrogenation catalyst is Co or Co oxide when the amine is a primary amine or Rh or Pt when the amine is an alkyl di-amine

4. The reactor system for dehydrogenating primary aliphatic or di-amines to their corresponding nitriles of claim 1, wherein the outer chamber or the inner reactor is surrounded by a resistive heating elements to provide sufficient temperature to catalyze the dehydrogenation reaction.

5. The reactor system for dehydrogenating primary aliphatic or di-amines to their corresponding nitriles of claim 1, wherein the alkyl di-amine is selected from the group consisting of 2-(aminomethyl)propane-1,3-diamine, propane-1,3-diamine, propylamine, ethylamine, butyl amine, propane-1,3-diamine, ethane-1,3-diamine, butane-1,3-diamine, pentane-1,3-diamine, isopropyl-1,3-diamine, and combinations thereof.

6. A reactor system for dehydrogenating primary aliphatic mono- or di-amines to their corresponding nitriles, comprising:
(a) a flow-through reactor having a circumferential outer reactor located surrounding an inner chamber;
(b) an outer circumferential reactor comprising a catalyst bed, an inlet and an outlet, and having an inner wall composed of a hydrogen membrane in that portion of the outer circumferential reactor located surrounding the inner chamber, and an outer wall of an impermeable material in that portion of the inner reactor located outside of the inner chamber, wherein the inlet further comprises a means for vaporizing a liquid to form a gaseous state prior to entering the catalyst bed;
(c) an inner chamber having an outlet and outer walls, wherein the outer walls are the inner walls of the circumferential reactor, and wherein the outlet further comprises a vacuum to pull through purified hydrogen formed in the outer circumferential reactor.

7. The reactor system for dehydrogenating primary aliphatic mono- or di-amines to their corresponding nitriles of claim 6, wherein the dehydrogenation catalyst in the outer circumferential reactor is selected from the group consisting of heterogeneous or homogeneous Group VIII metals, Rh, Pt, Ru, Au, Pd, cobalt, cobalt oxide, iron oxide, nickel oxide, chromium oxide, alloys of the foregoing and combinations thereof.

8. The reactor system for dehydrogenating primary aliphatic mono- or di-amines to their corresponding nitriles of claim 6, wherein the outer circumferential reactor is surrounded by a resistive heating element to provide sufficient temperature to catalyze the dehydrogenation reaction.

9. The reactor system for dehydrogenating primary aliphatic or di-amines to their corresponding nitriles of claim 6, wherein the alkyl di-amine is selected from the group consisting of 2-(aminomethyl)propane-1,3-diamine, propane-1,3-diamine, propylamine, ethylamine, butyl amine, propane-1,3-diamine, ethane-1,3-diamine, butane-1,3-diamine, pentane-1,3-diamine, isopropyl-1,3-diamine, and combinations thereof.

10. A process for dehydrogenating an aliphatic mono- or di-amine to its corresponding mono mono- and di-nitriles, comprising:
(a) providing a mono- and di-amine or a mixture thereof in a vapor form to a reactor having a dehydrogenation catalyst, an inlet and an outlet, wherein the mono- or di-amine or the mixture thereof is provided through the inlet and wherein the dehydrogenation catalyst is selected from the group consisting of Rh, Pt, Ru, Au, Pd, cobalt, cobalt oxide, iron oxide, nickel oxide, chromium oxide, alloys of the foregoing and combinations thereof;
(b) providing sufficient heat to dehydrogenate the mono- or di-amine into its corresponding mono- or di-nitriles and hydrogen gas;
(c) physically removing the hydrogen gas through a fractionating hydrogen membrane or by utilizing an inert sweep gas; and
(d) recovering the mono- or di-nitriles and the mixtures thereof formed by condensing the vapor in the outlet and recovering condensed liquid.

11. The process for dehydrogenating an aliphatic mono- or di-amine to its corresponding mono- and di-nitriles of claim 10, wherein the aliphatic di-amine is selected from the group consisting of 2-(aminomethyl)propane-1,3-diamine, propane-1,3-diamine, propylamine, ethylamine, butyl amine, propane-1,3-diamine, ethane-1,3-diamine, butane-1,3-diamine, pentane-1,3-diamine, isopropyl-1,3-diamine, and combinations thereof.

12. The process for dehydrogenating an aliphatic mono- or di-amine to its corresponding mono- and di-nitriles of claim 10, wherein the sweep gas is selected from the group consisting of He, Ar, and combinations thereof.
